SEQUENCE LISTING (23 sequences)

SEQ ID NO: 1 - DNA, 1997 bp, Brachypodium distachyon
agatttgtgattgttcagctacatgctccttgaagcacctttattctactataaattagttcgttggacgagactattagaccacatgctagccatgcttgttcgtagttgtcttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttca

SEQ ID NO: 2 - DNA, 684 bp, Artificial Sequence
  cDNA sequence  of the Zea mays AN3 gene
atgcagcagcaacacctgatgcagatgaaccagaacatgatggggggctacacctctcctgccgccgtgaccaccgatctcatccagcagtacctggacgagaacaagcagctgatcctggccatcctcgacaaccagaacaatggcaaggcggaggagtgcgaacggcaccaagctaagctccagcacaacctcatgtacctggccgccatcgctgacagccagccgccacagaccgcgccactatcacagtacccgtccaacctgatgatgcagccgggccctcggtacatgccaccgcagtccgggcagatgatgaacccgcagtcgctgatggcggcgcggtcctccatgatgtacgcgcacccgtccctgtcgccactccagcagcagcaggcggcgcacggacagctgggtatggctccagggggcggcggtggcggcacgaccagcgggttcagcatcctccacggcgaggccagcatgggcggtggtggtgctggcgcaggcgccggcaacaacatgatgaacgccggcatgttctcgggctttggccgcagcggcagtggcgccaaggaagggtcgacctctctgtcggttgacgtccggggtggaaccagctccggcgcgcagagcggggacggcgagtacctcaaagtcggcaccgaggaagaaggcagttag

SEQ ID NO: 3 - DNA, 1994 bp, Zea mays
gactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaa

SEQ ID NO: 4 - protein, 12 aa, Artificial Sequence
  AN3 gene
  MISC_FEATURE (3)..(3): X is K or Q
  MISC_FEATURE (10)..(10): X is K or Q or S
IQXYLDENKXLI

SEQ ID NO: 5 - protein, 15 aa, Artificial Sequence
  AN3 gene
  MISC_FEATURE (3)..(3): X is K, Q, H or R
  MISC_FEATURE (6)..(6): X is M or L
  MISC_FEATURE (14)..(14): X is A, T or S
LQXNLXYLAAIADXQ

SEQ ID NO: 6 - protein, 195 aa, Arabidopsis thaliana
MQQQQSPQMFPMVPSIPPANNITTEQIQKYLDENKKLIMAIMENQNLGKLAECAQYQALLQKNLMYLAAIADAQPPPPTPGPSPSTAVAAQMATPHSGMQPPSYFMQHPQASPAGIFAPRGPLQFGSPLQFQDPQQQQQIHQQAMQGHMGIRPMGMTNNGMQHAMQQPETGLGGNVGLRGGKQDGADGQGKDDGK

SEQ ID NO: 7 - protein, 223 aa, Arabidopsis thaliana
MQQSPQMIPMVLPSFPPTNNITTEQIQKYLDENKKLIMAILENQNLGKLAECAQYQALLQKNLMYLAAIADAQPQPPAATLTSGAMTPQAMAPNPSSMQPPPSYFMQQHQAVGMAQQIPPGIFPPRGPLQFGSPHQFLDPQQQLHQQAMQGHMGIRPMGLNNNNGLQHQMHHHETALAANNAGPNDASGGGKPDGTNMSQSGADGQGGSAARHGGGDAKTEGK

SEQ ID NO: 8 - protein, 210 aa, Arabidopsis thaliana
MQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQQQQQPYATLQHQQLHHSQLGMSSSSGGGGSSGLHILQGEAGGFHDFGRGKPEMGSGGGGEGRGGSSGDGGETLYLKSSDDGN

SEQ ID NO: 9 - protein, 229 aa, Oryza sativa
MQQQHLMQMNQGMMGGYASPTTVTTDLIQQYLDENKQLILAILDNQNNGKVEECARNQAKLQHNLMYLAAIADSQPPQTAAMSQYPSNLMMQSGARYMPQQSAQMMAPQSLMAARSSMMYAQPALSPLQQQQQQAAAAHGQLGMGSGGTTSGFSILHGEASMGGGGGGGGAGNSMMNAGVFSDFGRGGGGGGSGGKEGSTSLSVDVRGANSGAQSGDGEYLKGTEEEGS

SEQ ID NO: 10 - protein, 205 aa, Oryza sativa
MQQQMAMPAGAAAAAVPPAAGITTEQIQKYLDENKQLILAILENQNLGKLAECAQYQAQLQKNLLYLAAIADAQPPQNPGSRPQMMQPGATPGAGHYMSQVPMFPPRTPLTPQQMQEQQQQQLQQQQAQALAFPGQMLMRPGTVNGMQSIPVADPARAADLQTAAPGSVDGRGNKQDATSEPSGTESHKSAGADNDAGGDIAEKS

SEQ ID NO: 11 - protein, 185 aa, Oryza sativa
MQQQPMPMPAQAPPTAGITTEQIQKYLDENKQLILAILENQNLGKLAECAQYQAQLQKNLLYLAAIADTQPQTTISRPQMVPHGASPGLGGQYMSQVPMFPPRTPLTPQQMQEQQLQQQQAQLLSFGGQMVMRPGVVNGIPQLLQGEMHRGADHQNAGAATSEPSESHRSTGTENDGGSDFGDQS

SEQ ID NO: 12 - protein, 200 aa, Triticum aestivum
MQQAMSLPPGAVGAVSSPAGITTEQIQKYLDENKQLILAILENQNLGKLAECAQYQAQLQKNLLYLAAIADAQPPQNPTSHPQMVQPGSMQGAGHYMSQVPMFPPRTPLTPQQMQEQQHQQLQQQQAQALSFPAQVVMRPGTVNGMQQPMQAAGDLQPAAAPGGSKQDAAVAGASSEPSGTKSHKNAGAEEVGADVAEQS

SEQ ID NO: 13 - protein, 226 aa, Triticum aestivum
MQQQHLMQMNQSMMGGYASSTTVTTDLIQQYLDENKQLILAILDNQNNGKVEECARNQAKLQQNLMYLAAIADSQPPQTASLSQYPSNLMMQSGPRYMPQQSAQMMSPQSLMAARSSMMYAQQAMSPLQQQQQQQQQHQAASHGQLGMSSGATTGFNLLHGEASMGGGGGATGNSMMNAGVFSDYGRGGSGAKEGSTSLSADARGANSGAHSGDGEYLKGTEEEGS

SEQ ID NO: 14 - protein, 225 aa, Triticum aestivum
MQQQHLMQMNQSMMGGYASSTTVTTDLIQQYLDENKQLILAILDNQNNGKVEECARNQAKLQQNLMYLAAIADSQPPQTASLSQYPSNLMMQSGPRYMQQQSAQMMSPQSLMAARSSMMYAQQAMSPLQQQQQQQHHQAAAHGQLGMSSGATTGFNLLHGEASMGGGGGATGNSMMNASVFSDYGRGGSGAKEGSTSLSADARGANSGAHSGDGEYLKGTEEEGS

SEQ ID NO: 15 - protein, 195 aa, Triticum aestivum
MQQAMSLPPGAVGAVSPPAGITTEQIQKYLDENKQLILAILENQNLGKLAECAQYQAQLQKNLLYLAAIADAQPPQNPASRPQMVQQPGSMQGAGHYMSQVPMFPPRTPLTPQQMQEQQQAQALSFPAQMVMRPGTINGMQLPMQAAGDLQPAAAPGGSKQDAVVAGASSEPSGTESHKKAGAEKEVSADVAEQS

SEQ ID NO: 16 - protein, 225 aa, Triticum aestivum
MQQQHLMQMNQSMMGGYASSTTVTTDLIQQYLDENKQLILAILDNQNNGKVEECARNQAKLQQNLMYLAAIADSQPPQTASLSQYPSNLMMQSGPRYMPQQSAQMMSPQSLMAARSSMMYAQQAMSPLQQQQQQQHQAAAHGQLGMSSGATTGFNLLHGEASMGGGGGGASGNSMMNAGVFSDYGRGGSGAKEGSTSLSADARGANSGAHSGDGEYLKGTEEEGS

SEQ ID NO: 17 - protein, 185 aa, Triticum aestivum
MQQAMPMPPAAAAPGMPPSAGLSTEQIQKYLDENKQLILAILENQNLGKLAECAQYQAQLQKNLLYLAAIADTQPQTTVSRPQMAPPSASPGAGHYMSQVPMFPPRTPLTPQQMQEQQLQQQQAQMLPFAGQMVARPGAVNGMPQAPQVEPAYAAGGASSEPSGTESHRSTGADNDGGSGLADQS

SEQ ID NO: 18 - protein, 185 aa, Triticum aestivum
MQQAMPMPPAAAAPGMPPSAGLSTEQIQKYLDENKQLILAILENQNLGKLAECAQYQAQLQKNLLYLAAIADTQPQTSVSRPQMAPPSASPGVGHYMSQVPMFPPRTPLTPQQMQEQQLQQQQAQMLPFAGQMVARPGAVNGMARAPQVEPAYAAGGASSEPSGTESHRSTGADNNGGSGLADQS

SEQ ID NO: 19 - protein, 185 aa, Triticum aestivum
MQQAMPMPAAAAAPGMPPSAGLSTEQIQKYLDENKQLILAILENQNLGKLAECAQYQAQLQKNLLYLAAIADTQPQTSVSRPQMAPPSASPGAGHYMSQVPMFPPRTPLTPQQMQEQQLQQQQAQMLPFAGQMVVRPGAVNGMPHAPQVEPAYAAGGASSEPSGTESHRSTGADNDGGSGLADQS

SEQ ID NO: 20 - protein, 182 aa, Zea mays
MQQPMHMQPQAPAITPAAGISTEQIQKYLDENKQLILAILENQNLGKLAECAQYQSQLQKNLLYLAAIADAQPQTAVSRPQMAPPGGSPGVGQYMSQVPMFPPRTPLTPQQMQEQQLQQQQAQLLNFSGQMVARPGMVNGMAQSMQAQLPPGVNKQDAGGVASEPSGTESHRSTGGDDGGSD

SEQ ID NO: 21 - protein, 227 aa, Zea mays
MQQQHLMQMNQNMMGGYTSPAAVTTDLIQQYLDENKQLILAILDNQNNGKAEECERHQAKLQHNLMYLAAIADSQPPQTAPLSQYPSNLMMQPGPRYMPPQSGQMMNPQSLMAARSSMMYAHPSLSPLQQQQAAHGQLGMAPGGGGGGTTSGFSILHGEASMGGGGAGAGAGNNMMNAGMFSGFGRSGSGAKEGSTSLSVDVRGGTSSGAQSGDGEYLKVGTEEEGS

SEQ ID NO: 22 - protein, 183 aa, Zea mays
MPMQPQAPSMTPAAGITTEQIQKYLDENKQLILAILENQNLGKLAECAQYQSQLQKNLLYLAAIADAQPETAVSRPQMAPPGASPGVGQYMSQVPMFPPRTPLTPQQMLEQQLQQQQAQLLNFSGQMVARPGMVNGMLQAMQVQQAQPSPVMNRPDAGGIASEPSGTESHRTSTGGDNGGGSD

SEQ ID NO: 23 - protein, 220 aa, Zea mays
MQQQMPMPPAPAAAAAAAPPAAGITTEQIQKYLDENKQLILAILENQNLGKLAECAQYQAQLQKNLLYLAAIADAQPQPPQNPAGRPQMMQPGIVPGAGHYMSQVPMFPPRTPLTPQQMQEQQQQQQFQQQQQQVQALTFPGQMVMRPGTINGMQQQQPMQADPARAAAELQQAAPIPADGRGSKQDTAGGASSEPSANESHKSATGADTEAGGDVAEKS

(19) United States
(12) Patent Application Publication
(10) Pub. No.: US 2024/0318194 A1
(43) Pub. Date: Sep. 26, 2024

(54) MEANS AND METHODS FOR PRODUCING DROUGHT TOLERANT CEREALS

(71) Applicants: VIB VZW, Gent (BE); UNIVERSITEIT GENT, Gent (BE)

(72) Inventors: Dirk Gustaaf Inzé, Moorsel - Aalst (BE); Hilde Nelissen, Gent (BE); Reinout Laureyns, Gent (BE)

(21) Appl. No.: 18/260,975

(22) PCT Filed: Jan. 11, 2022
(86) PCT No.: PCT/EP2022/050370

§ 371 Date: Jul. 11, 2023

(30) Foreign Application Priority Data
Jan. 12, 2021 (EP) 21151186.0

(51) Int. Cl. C12N 15/82; C07K 14/415

(52) U.S. Cl. CPC C12N 15/8273; C07K 14/415; C12N 15/8261

(57) ABSTRACT

The present invention relates to the field of plant molecular biology, more particularly to the field of agriculture, even more particularly to the field of improving the yield of plants. The present invention provides chimeric genes and constructs which can be used to enhance the yield in plants and crops.

### CROSS REFERENCE TO RELATED APPLICATIONS

This application is a national phase entry under 35 U.S.C. § 371 of International Patent Application PCT/EP2022/050370, filed Jan. 11, 2022, designating the United States of America and published in English as International Patent Publication WO 2022/152660 on Jul. 21, 2022, which claims the benefit under Article 8 of the Patent Cooperation Treaty to European Patent Application Serial No. 21151186.0, filed Jan. 12, 2021, the entireties of which are hereby incorporated by reference.

### FIELD OF THE INVENTION

The present invention relates to the field of plant molecular biology, more particularly to the field of agriculture, even more particularly to the field of improving the yield of plants. The present invention provides chimeric genes and constructs which can be used to enhance the yield and drought tolerance in crops such as cereals.

Introduction to the invention Since the beginning of agriculture and horticulture, there was a need for improving plant traits in crop cultivation. Breeding strategies foster crop properties to withstand biotic and abiotic stresses, to improve nutrient use efficiency and to alter other intrinsic crop specific parameters, i.e. increasing yield by applying technical advances. In the coming decades, a crucial challenge for humanity will be meeting future food demands without undermining further the integrity of the Earth's environmental systems. Agricultural systems are already major forces of global environmental degradation, but population growth and increasing consumption of calorie- and meat-intensive diets are expected to roughly double human food demand by 2050. Responding to these pressures, there is increasing focus on ‘sustainable intensification’ as a means to increase yields on underperforming landscapes while simultaneously decreasing the environmental impacts of agricultural systems. Conventional means for crop and horticultural improvements today utilize selective breeding techniques to identify plants with desirable characteristics. Advances in molecular biology have allowed to modify the germplasm of plants in a specific way. For example, the modification of a single gene resulted in several cases in a significant increase in yield or yield-related traits.

Angustifolia3 (AN3), also called GRF-Interacting Factor 1 (GIF1), encodes a putative transcriptional coactivator with homology to human synovial sarcoma translocation protein. AN3 transcripts accumulate in mesophyll cells but are not detectable in leaf epidermal cells. AN3 moves into epidermal cells after being synthesized within mesophyll cells and helps control epidermal proliferation. Interference with AN3 movement results in abnormal leaf size and shape, indicating that AN3 movement indispensable for normal leaf development.

Recently, the mode of action of the Arabidopsis thaliana leaf growth-regulating protein ANGUSTIFOLIA3/GRF-INTERACTING FACTOR1 (AN3/GIF1), a transcriptional coactivator lacking direct DNA binding properties, was elucidated by the identification of its interacting partners through tandem affinity purification (TAP) followed by mass spectrometry. AN3/GIF1 physically interacts with chromatin remodeling complexes containing the ATPases SPLAYED (SYD) and BRAHMA (BRM). TAP using AN3/GIF1 as bait identified novel players important for leaf growth, which was validated by the observation that some of these interacting proteins displayed growth-enhancing phenotypes when their expression was modified (Vercruyssen L. et al (2014) Plant Cell 26, 210-229). In addition, using yeast two-hybrid and immunoprecipitation assays, AN3 was shown to interact with GROWTH-REGULATING FACTOR (GRF) proteins, a class of plant-specific transcriptional activators of which some members are posttranscriptionally regulated by microRNA396a. It has further been shown in the art that plants lacking AN3 activity have high drought stress tolerance because of low stomatal densities and improved root architecture (see Lai-Sheng Meng and Shun-Qiao Yao (2015) Plant Biotechnology Journal 13, 893-902).

The maize AN3 coding sequence was previously expressed in corn plants under the control of the weak constitutive maize UBI-L promoter (Coussens G. et al (2012) Journal of Experimental Botany 63, 4263-4273). These transgenic corn plants did not show an increase in final leaf size or an altered leaf elongation rate and it was observed that the plants developed more slowly than the non-transgenic siblings (see Nelissen H. et al. (2015) The Plant Cell 27, 1605-1619). In the present invention it is surprisingly shown that expression of the coding sequence of the corn AN3 gene under control of the Brachypodium distachyon EF1-alpha promoter (Coussens G. et al (2012) Journal of Experimental Botany 63, 4263-4273) leads to an enhanced yield and drought tolerance in corn plants and that the transgenic plants have no developmental growth effects.

### FIGURES

FIG. 1: Ear leaf width of two independent AN3 overexpressing lines grown in Belgian field trials in 2020. S=non-transgenic and R=transgenic

FIG. 2: Stem width (maximal) of two independent AN3 overexpressing lines grown in Belgian field trials in 2020. S=non-transgenic and R=transgenic

FIG. 3: Stem width (minimal) of two independent AN3 overexpressing lines grown in Belgian field trials in 2020. S=non-transgenic and R=transgenic

FIG. 4: Ear dry weight of two independent AN3 overexpressing lines grown in Belgian field trials in 2020. WT=non-transgenic and T=transgenic

FIG. 5: Stem dry weight of two independent AN3 overexpressing lines grown in Belgian field trials in 2020. WT=non-transgenic and T=transgenic

FIG. 6: Leaf dry weight of two independent AN3 overexpressing lines grown in Belgian field trials in 2020. WT=non-transgenic and T=transgenic

FIGS. 7A and 7B: Alignment AN3/GIF1 plant orthologous protein sequences. Two conserved boxes are underlined and in bold

FIG. 8: we compared the expression level of GIF1 in the BdEF1a::GIF1-07 line with the ZmUBIL::GIF1 line. A clear distinction could be made between the line overexpressing GIF1 using the Bd EF1alpha promoter and the Zm UBIL promoter. For both lines the non-transgenic sibling was used as negative control.

FIG. 9: lamina length of corn plants transformed with the chimeric gene (T) is increased under well-watered (WW) and water-deficit (WD) conditions as compared to the wild type corn plants (NT). Experiment was carried out in an automatic greenhouse-based phenotyping platform.

FIG. 10: ear weight of corn plants transformed with the chimeric gene (T) is increased under water-deficit (WD) conditions as compared to the wild type corn plants (NT). Experiment was carried out in an automatic greenhouse-based phenotyping platform.

FIG. 11: Results from the 2021 field trial in Belgium of the EF1a::AN3_07 transgenic and non-transgenic plants, as average of the three independent plots. Error bars represent standard deviation.

### DETAILED DESCRIPTION OF THE INVENTION

To facilitate the understanding of this invention a number of terms are defined below. Terms defined herein (unless otherwise specified) have meanings as commonly understood by a person of ordinary skill in the areas relevant to the present invention. As used in this specification and its appended claims, terms such as “a”, “an” and “the” are not intended to refer to only a singular entity but include the general class of which a specific example may be used for illustration, unless the context dictates otherwise. The terminology herein is used to describe specific embodiments of the invention, but their usage does not delimit the invention, except as outlined in the claims.

In the present invention we have surprisingly shown that a chimeric gene comprising the EF1A promoter of Brachypodium dystachyon gene operably coupled to the nucleotide sequence of a plant AN3 gene, when introduced or transformed into a plant leads to an enhanced yield and an enhanced drought tolerance.

The promoter region of the Elongation factor 1-alpha gene (EF1A gene) from Brachypodium distachyon is described in Coussens G. et al (2012) Journal of Experimental Botany 63, 4263-4273) and was shown to be active in Zea mays. The sequence of the EF1A promoter is depicted in SEQ ID NO: 1. It is understood that slightly shorter or slightly longer versions of SEQ ID NO: 1 can be used in the context of the present invention.

Accordingly, in a first embodiment the invention provides a chimeric gene construct comprising the following operably linked DNA elements: a) the promoter region of a the Brachypodium distachyon EF1A gene, b) a DNA region encoding a plant AN3 protein and c) a 3′ end region comprising transcription termination and polyadenylation signals functioning in cells of a plant.

The AN3 gene is well known in plants (see example 4 for orthologous sequences). In the present invention AN3 is interchangeably used with GIF, GIF1, GIF2 or GIF3.

In a particular embodiment the cDNA sequence of the corn AN3 gene is depicted in SEQ ID NO: 2.

In another particular embodiment the protein sequence of the AN3 gene comprises SEQ ID NO: 4 and SEQ ID NO: 5.

It is understood that a particular chimeric gene can be used as a trait in different plant species and that the Brachypodium distachyon (B d) EF1A promoter is active in more than one plant species.

In a specific embodiment the Brachypodium distachyon promoter is active in cereals such as maize (corn), wheat, rice, oats, barley, rye, millet or sorghum.

The term “operably linked” as used herein refers to a functional linkage between the promoter sequence (here the Brachypodium distachyon EF1A promoter) and the gene of interest (here the AN3 gene), such that the EF1A promoter sequence is able to initiate transcription of the AN3 gene of interest.

A “chimeric gene” or “chimeric construct” is a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not normally operatively linked to the associated nucleic acid sequence as found in nature.

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

In yet another embodiment the invention provides a recombinant vector comprising a chimeric gene construct comprising the following operably linked DNA elements: a) the promoter region of the Brachypodium distachyon EF1A gene, b) a DNA region encoding a plant AN3 gene and c) a 3′ end region comprising transcription termination and polyadenylation signals functioning in cells of a plant.

In yet another embodiment the invention provides a plant, plant cell or plant seed comprising a chimeric gene construct comprising the following operably linked DNA elements: a) the promoter region of the Brachypodium distachyon EF1A gene, b) a DNA region encoding a plant AN3 gene and c) a 3′ end region comprising transcription termination and polyadenylation signals functioning in cells of a plant or a recombinant vector comprising a chimeric gene construct comprising the following operably linked DNA elements: a) the promoter region of the Brachypodium distachyon EF1A gene, b) a DNA region encoding a plant AN3 gene and c) a 3′ end region comprising transcription termination and polyadenylation signals functioning in cells of a plant.

In yet another embodiment the invention provides the use of a chimeric gene or a recombinant vector according to the invention increase the yield of plants.

In yet another embodiment the invention provides the use of a chimeric gene or a recombinant vector according to the invention to increase the drought tolerance of plants. In a specific embodiment the chimeric gene or recombinant vector comprising the chimeric gene of the invention is used to increase the drought tolerance of cereals such as for example corn.

In a specific embodiment the chimeric genes or recombinant vector comprising the chimeric genes are used in crops.

In another specific embodiment crops are cereals.

In yet another specific embodiment crops are grasses.

In yet another embodiment the invention provides a method to produces a plant with increased yield as compared to a corresponding wild type plant, whereby the method comprises introducing or transforming a chimeric gene or a recombinant vector according to the invention.

In yet another particular embodiment the chimeric gene of the invention is combined with other chimeric genes which favorable increase the yield of plants. A particular example is the combination of the chimeric GA2ox promoter-KLUH gene as disclosed in WO2014195287.

The term “yield” as used herein generally refers to a measurable product from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable. As used herein, the term “improved yield” or the term “increased yield” means any improvement in the yield of any measured plant product, such as grain, fruit, leaf, root, cob or fiber. In accordance with the invention, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, and/or higher dry matter production. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the chimeric genes of the invention, as compared with the bu/acre yield from untransformed soybeans or corn cultivated under the same conditions, is an improved yield in accordance with the invention. The increased or improved yield can be achieved in the absence or presence of stress conditions. For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both. “Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), harvestable parts weight per area (acre, square meter, or the like); and the like. The harvest index is the ratio of yield biomass to the total cumulative biomass at harvest. Harvest index is relatively stable under many environmental conditions, and so a robust correlation between plant size and grain yield is possible. Measurements of plant size in early development, under standardized conditions in a growth chamber or greenhouse, are standard practices to measure potential yield advantages conferred by the presence of a transgene. Accordingly, the yield of a plant can be increased by improving one or more of the yield-related phenotypes or traits. Such yield-related phenotypes or traits of a plant the improvement of which results in increased yield comprise, without limitation, the increase of the intrinsic yield capacity of a plant, improved nutrient use efficiency, and/or increased stress tolerance. For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like). “Yield” can also refer to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture. For example, the term “increased yield” means that a plant, exhibits an increased growth rate, e.g. in the absence or presence of abiotic environmental stress, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds. A prolonged growth comprises survival and/or continued growth of the plant, at the moment when the non-transformed wild type organism shows visual symptoms of deficiency and/or death. When the plant of the invention is a corn plant, increased yield for corn plants means, for example, increased seed yield, in particular for corn varieties used for feed or food. Increased seed yield of corn refers to an increased kernel size or weight, an increased kernel per ear, or increased ears per plant. Alternatively, or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant. When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant. When the plant of the invention is a cotton plant, increased yield for cotton plants means increased lint yield. Increased lint yield of cotton refers in one embodiment to an increased length of lint. When the plant is a plant belonging to grasses an increased leaf can mean an increased leaf biomass. Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance. Intrinsic yield capacity of a plant can be, for example, manifested by improving the specific (intrinsic) seed yield (e.g. in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like. “Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta©; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example p-glucuronidase, GUS or β-galactosidase with its coloured substrates, for example X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the IoxP sequences. If the marker gene is integrated between the IoxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention.

A transgenic plant for the purposes of the invention is thus understood as meaning, as above, that the nucleic acids used in the method of the invention are not present in, or originating from, the genome of said plant, or are present in the genome of said plant but not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, heterologous expression of the nucleic acids takes place. Preferred transgenic plants are mentioned herein.

For the purpose of this invention related or orthologous genes of the AN3 gene as described herein before can be isolated from the (publicly) available sequence databases. The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453) The computer-assisted sequence alignment above, can be conveniently performed using standard software program such as GAP which is part of the Wisconsin Package Version 10.1 (Genetics Computer Group, Madison, Wisconsin, USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical.

Alternatively, the skilled person can isolate orthologous plant AN3 genes through methods of genetic hybridization. Such methods are well known to the skilled (plant) molecular biologist.

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1 102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP1198985, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al (1984) Nucl. Acids Res. 12-8711).

Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, KA and Marks M D (1987). Mol Gen Genet 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). CR Acad Sci Paris Life Sci, 316: 1 194-1 199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally, after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35%, 40%, 50%, 60%, 70%, 80%, 90%, 95% or more yield and/or growth in comparison to control plants as defined herein.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

The term “expression cassette” refers to any recombinant expression system for the purpose of expressing a nucleic acid sequence of the invention in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e., drive only transient expression in a cell). The term includes recombinant expression cassettes that contain only the minimum elements needed for transcription of the recombinant nucleic acid.

The following non-limiting Examples describe methods and means according to the invention. Unless stated otherwise in the Examples, all techniques are carried out according to protocols standard in the art. The following examples are included to illustrate embodiments of the invention. Those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### EXAMPLES

1. Construction of a Chimeric Gene: Brachypodium distachyon EF1alpha Promoter Operably Linked to a AN3 Coding Sequence

The Brachypodium distayon EF1alpha promoter was isolated (depicted in SEQ ID NO: 1) and fused with attB4 and attB1r sites and combined with entry vector pDONR P4-P1r by BP reaction.

A representative corn AN3 gene was also isolated (cDNA sequence is depicted in SEQ ID NO: 2). The AN3 sequence was fused with attB1 and attB2 sites and combined with entry vector pDONR 221 by BP reaction.

Expression vector pBb42GW7 is a MultiSite Gateway intermediary vectors designed for monocot (Karimi, M. et al (2013) Trends in Plant Science 18, 1-4). B. distachyon EF1alpha promoter operably linked to the corn AN3 gene was inserted into expression vector pBb42GW7 through LR reaction between attR4 and attR2. Bar gene driven by 35S promoter was used for selecting transgenic plants during the transformation process.

Maize transformation was performed according to Coussens G. et al (2012) Journal of Experimental Botany 63, 4263-4273.

In total, 10 independent TO lines were obtained after transformation. Around 35 T1 seeds from TO backcrossed with wild type B104 were sown in soil for segregation analysis and phenotyping. Ammonium assay (De Block et al (1995) Planta 197, 619-626) was used to detect transgenic plants, leaf painting was used to confirm certain plants for upscaling.

Two independent lines (line 05 and line 07) which have one T-DNA insertion were selected for phenotyping.

### 2. Field Trial Data of Corn Transgenic Lines Comprising the Chimeric Gene of Example 1

### 2.1 Field Trial 2020:

Two independent lines (line 05 and line 07 of which line 07 has the highest level of AN3 overexpression) with the respective non-transgenic controls were sown in triplicate on May 14, 2020 and harvested on Oct. 27, 2020. Due to early drought early in the season there was a low germination rate. It was apparent that a higher percentage (+/−22-24%) of the transgenic plants germinated relative to the non-transgenic controls during the drought spell. Several parameters were found to be significant for line 07 but not for line 05.

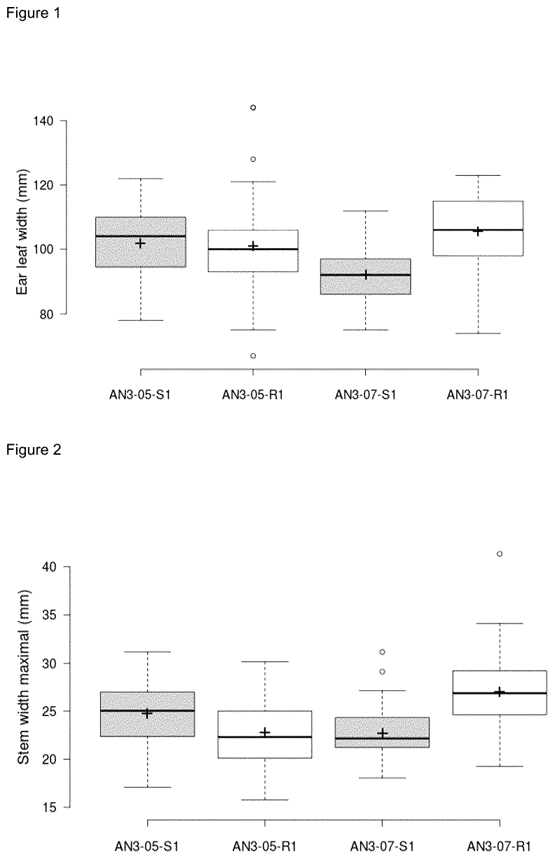

The ear leaf width was significantly increased in the transgenics of line 07 (see FIG. 1).

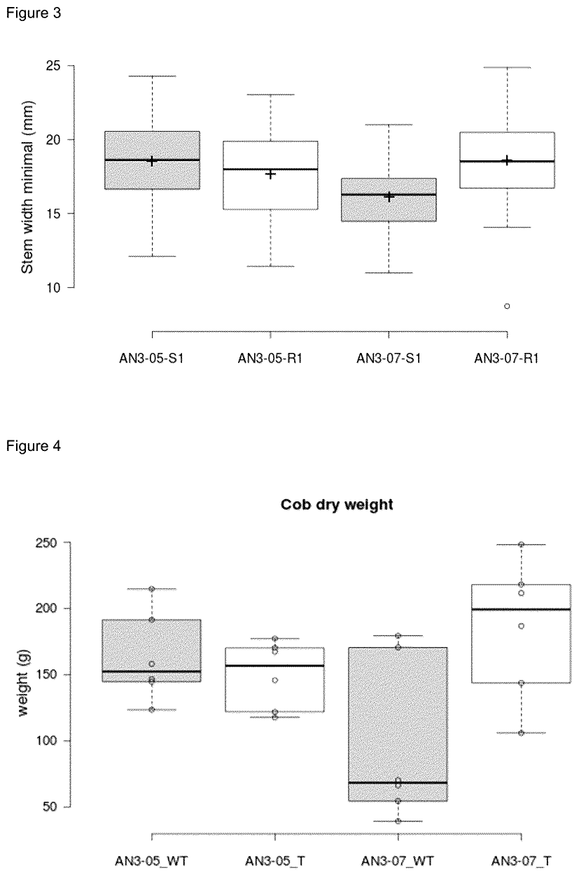

The stem width was significantly increased in the transgenics of line 07 (in both directions, as the stem is oval shaped; see FIG. 2 and FIG. 3)

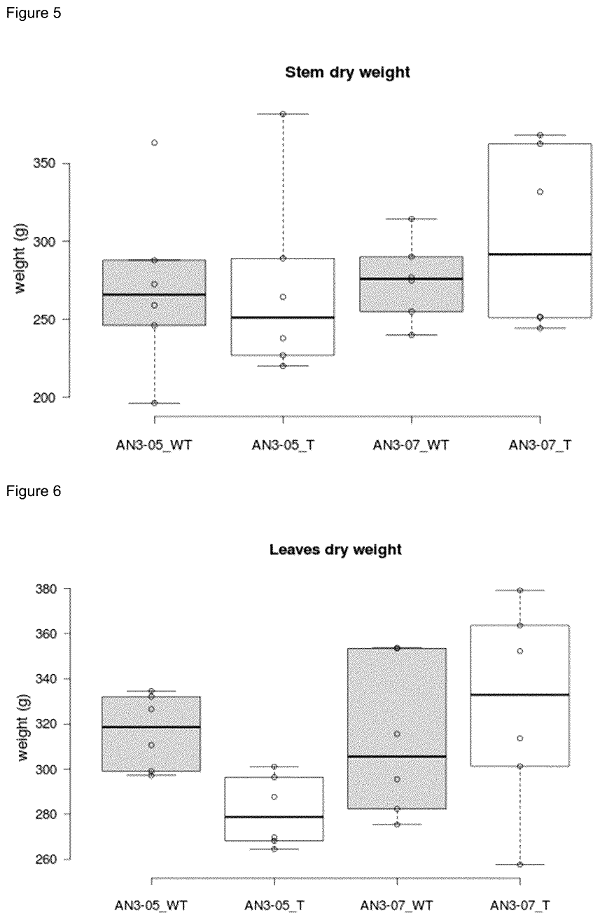

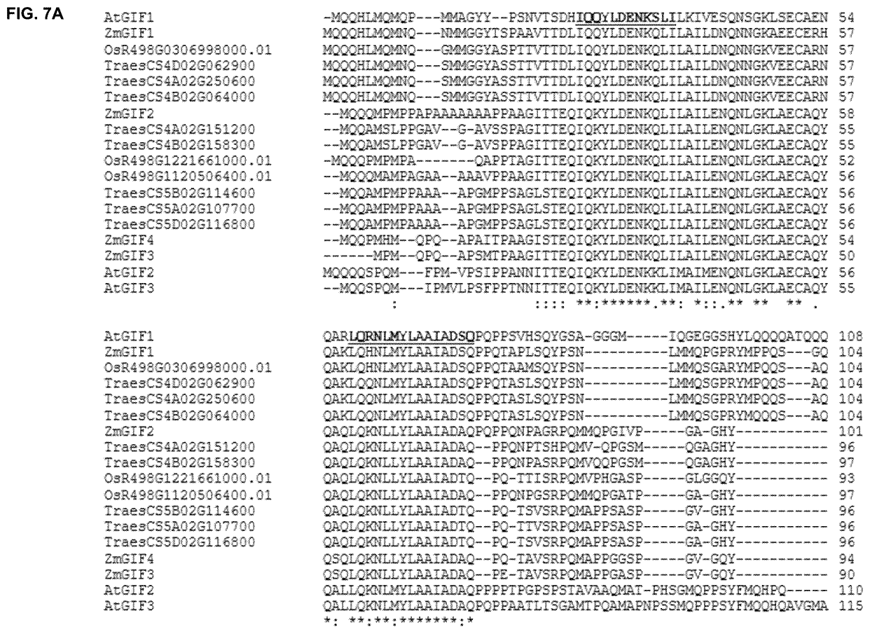

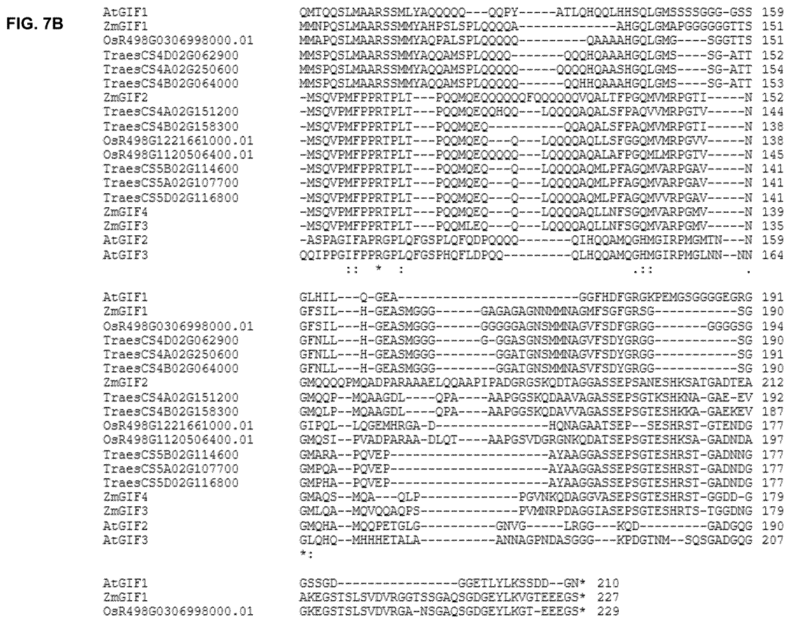

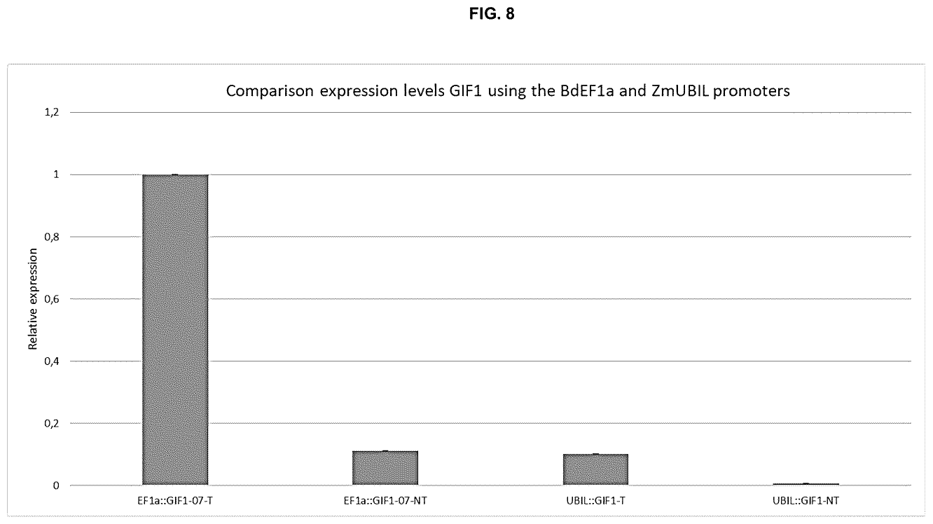

With respect to biomass, there was an increase in dry weight of the ear (see FIG. 4), stem (see FIG. 5) and leaves (see FIG. 6) of line 07, however this was only significant for leaf and ear. For the ear, also the fresh weight was significantly higher in line 07.

Last but not least also the seed yield potential of the line 07 transgenic plants was greater than that of the respective non-transgenic plants because 15% more spikelets (p=0.015) were formed per row in line 07.

We conclude that despite the fact that 2020 was not the ideal field season (dry spring and relatively grey fall preventing full seed set), we observed surprising results for the highest overexpressing line 07 with respect to leaf and stem width and biomass, ear biomass, drought tolerance and seed yield potential. Importantly, no effect was seen on final plant height and no other aspects that could negatively affect yield (e.g. lodging or altered responsiveness to smut) were observed in the transgenic plants compared with the respective controls. The data show that high levels of AN3 are needed to result in promising phenotypes (as compared to the lack of these beneficial phenotypes when AN3 was expressed under control of the UBI-L promoter, depicted in SEQ ID NO: 3, see FIG. 8.

### 2.2 Field Trial 2021:

The same two lines (line 05 and line 07) with the respective non-transgenic controls were sown in triplicate on May 11, 2021 and harvested on Oct. 20, 2021. In 2021, the season was not characterized by extreme weather conditions and all four genotypes germinated properly and developed similarly. Upon harvest, line EF1a::AN3_07 again displayed increases in biomass and seed yield (see FIG. 11). An increase in both stem (19%, p-value 0.144) and leaf biomass (36%, p-value 0.054), resulting from increase in stem and leaf width, caused the significant increase in overall biomass (25%, p-value 0.063). An increased cob length (9.6%, p-value 4,95E-05) and 100 kernel weight (23%, p-value 2,15E-06) explained the seed yield gain. Similar to 2020, transgenic line EF1a::AN3_05 displayed no pronounced positive or negative phenotypes compared with the corresponding non-transgenic plants.

We conclude that line EF1a::AN3_07, resulted in higher biomass and seed yield in two subsequent field trials in Belgium. In addition, line EF1a::AN3_07 also germinated better during a dry start of the season. No differences were observed for flowering time or lodging as compared to the non-transgenic line.

### 3. Sequences

SEQ ID NO: 1
Promoter sequence of Brachypodium distachyon
AGATTTGTGATTGTTCAGCTACATGCTCCTTGAAGCACCTTTATT
CTACTATAAATTAGTTCGTTGGACGAGACTATTAGACCACATGCT
AGCCATGCTTGTTCGTAGTTGTCTTCACCGCCATTGCAAAAATTG
TCAATAAATATTTAGAGTGGGTGGCATCAGAAAAACATCTCTAGT
GGACTCTCTTCCTATCATAGCTACTCGGGCTGTAGATAGAACGAG
GGCACAAGAGTTGGGTGGCGTAGGTTTACTCGTGACCTCAACTCT
TTTGGCTGTGTCTTACGTCTAAGATGGGTTTGGCATGTGAGAAAC
ATAGGTCTAAGCAATTCATGTTAGGGCTGTTGCATTGTTGTTGCA
TCAACCAAATGTCCAGATAGCAGTTCATGCTACATCTAGTTGAAA
ACCCTCATCATTAGGCGGAACATGTGTTCTTTTTTAGCATAGTCA
AAGTCAGATTGCGGCACTCGCTCATCCACGGAAAGAATTTTCCCT
GTGCAGGCATCTCGATCAAAAGACGCAAATTAATTTTTGAATAGC
GATATAACAATATCTAATTAACGTTTCTTGTTTTCTGCGAAATGT
CTTTCATCATAAAATGAGTCATCTCGATGAGCCCAAGTGACATAG
CCCAACACCCCACCCCACCAATAAAAGTGAAGAAAACATGTTGGG
AAAACTATACCAAGTAAAATACGAGTTGTTCTAAAGAAAAAGTAA
AGTACGAGTTAGATCGCACCCTGTCCTGGAGTGTGGCTTGATGAT
CCAACTCCTAGCATTGTATCCCTGTTTTTGGATGATGTAACTATT
ATTTACAATGAATAAAGAGGTGTTTTACTAGTAAAAAAATCTTGA
GGGGAGGAGAAAATAATGGAGGTCTTTTTTCAAACCGATGGACTA
TTATTTTTAGTGAAAGAGAATAATATTATTGGAAAAATTATTCTA
TCCACTTATTTTATATTGGCAGAATACAAAGAATGGTGGGGTCCA
CGCGGAACTTGCGGCCCCCGAAACCTATCGAGGGCGCGGTACCCA
AGCAAGGAACGGAGGAAACTTGCGGGGCCCGAAACCTAGTGATAA
AAGGCATATCATCCACACGATGAAGATCTGACGGACCATATCTCC
CACCACGGAAAGCCATCAGACGAGGATCAGACGGCCAGGAAGGAA
CCCTAGCGCCCGCCGGTGCCAATATAAAGCGCCACTCTCTCTCGT
CTTAAGCCCCAGCCTCTCCATTCCCCTCTCCCTCTCGCCGCCGCC
GTCTCCTTCTCCTACTCCCTTCGAGGTGTGTTGTTCATCCGTCCC
GAATCCATCCATCCCCTCTTCAGATGTGTTGTTCATGGCTCTAAT
AGCTCTAGATCTGCTTGTTTGTGTTGTTTAGCTCTAGATCTACTC
GCGCGCGCTTCTCTCTCGATCTCCTGTAGAACAATTTTGGTTGGT
TTTTTGTGCATATCCATGGTAATTTTGTCTGCAATATGGAGGAGG
CTTTCTAAGCTCCTACGTAGCATCGATCTTTAGAATTCCCTCGGT
TTCTGTTTATTTCTTCGCGAGGGCTCTCTGTTATCTGTAGGAGTA
GCTGTAAGCGCGGTTCGTTACGGATTAATCGTCATGCTTAGTTGA
ACCTATCGGTCGAAGGATTTGTGTGGGTTGTCGTGTAGAATTGAC
ACCATCTACTTACTGTACTGATATGCCGATCTGTAGGATACTCTT
CATTACTTTTGTTTACTGCTAGTTGTGGTGTAGATTTAGCATTCT
CAAACCCATGCTGTAGCGTTTCTAATATTGTTACATAGATCTACC
GGTGCCTGTTAATTGTATTCGATCGGGCGTTTCTACATCTGTCCG
CCCACCTAGTTTTATATGTGGTAATCAAAATTGCGTTGACTTCGT
GATGCTGTCTGTGTACTGTTTTTAATCGCTCTTACTTAGATGATC
AACATGGTGATGGTTACGATTTACTGTTTTCTAATCCCTGTTACT
TCGATGCTGCAGTTTCA
SEQ ID NO: 2
cDNA sequence of the Zea mays AN3 gene
ATGCAGCAGCAACACCTGATGCAGATGAACCAGAACATGATGGGG
GGCTACACCTCTCCTGCCGCCGTGACCACCGATCTCATCCAGCAG
TACCTGGACGAGAACAAGCAGCTGATCCTGGCCATCCTCGACAAC
CAGAACAATGGCAAGGCGGAGGAGTGCGAACGGCACCAAGCTAAG
CTCCAGCACAACCTCATGTACCTGGCCGCCATCGCTGACAGCCAG
CCGCCACAGACCGCGCCACTATCACAGTACCCGTCCAACCTGATG
ATGCAGCCGGGCCCTCGGTACATGCCACCGCAGTCCGGGCAGATG
ATGAACCCGCAGTCGCTGATGGCGGCGCGGTCCTCCATGATGTAC
GCGCACCCGTCCCTGTCGCCACTCCAGCAGCAGCAGGCGGCGCAC
GGACAGCTGGGTATGGCTCCAGGGGGGGGCGGTGGCGGCACGACC
AGCGGGTTCAGCATCCTCCACGGCGAGGCCAGCATGGGCGGTGGT
GGTGCTGGCGCAGGCGCCGGCAACAACATGATGAACGCCGGCATG
TTCTCGGGCTTTGGCCGCAGCGGCAGTGGCGCCAAGGAAGGGTCG
ACCTCTCTGTCGGTTGACGTCCGGGGTGGAACCAGCTCCGGCGCG
CAGAGCGGGGACGGCGAGTACCTCAAAGTCGGCACCGAGGAAGAA
GGCAGTTAG
SEQ ID NO: 3
Promoter sequence of the UBI-L promoter of
Zea mays
GACTGCAGTGCAGCGTGACCCGGTCGTGCCCCTCTCTAGAGATAA
TGAGCATTGCATGTCTAAGTTATAAAAAATTACCACATATTTTTT
TTGTCACACTTGTTTGAAGTGCAGTTTATCTATCTTTATACATAT
ATTTAAACTTTACTCTACGAATAATATAATCTATAGTACTACAAT
AATATCAGTGTTTTAGAGAATCATATAAATGAACAGTTAGACATG
GTCTAAAGGACAATTGAGTATTTTGACAACAGGACTCTACAGTTT
TATCTTTTTAGTGTGCATGTGTTCTCCTTTTTTTTTGCAAATAGC
TTCACCTATATAATACTTCATCCATTTTATTAGTACATCCATTTA
GGGTTTAGGGTTAATGGTTTTTATAGACTAATTTTTTTAGTACAT
CTATTTTATTCTATTTTAGCCTCTAAATTAAGAAAACTAAAACTC
TATTTTAGTTTTTTTATTTAATAATTTAGATATAAAATAGAATAA
AATAAAGTGACTAAAAATTAAACAAATACCCTTTAAGAAATTAAA
AAAACTAAGGAAACATTTTTCTTGTTTCGAGTAGATAATGCCAGC
CTGTTAAACGCCGTCGACGAGTCTAACGGACACCAACCAGCGAAC
CAGCAGCGTCGCGTCGGGCCAAGCGAAGCAGACGGCACGGCATCT
CTGTCGCTGCCTCTGGACCCCTCTCGAGAGTTCCGCTCCACCGTT
GGACTTGCTCCGCTGTCGGCATCCAGAAATTGCGTGGCGGAGCGG
CAGACGTGAGCCGGCACGGCAGGCGGCCTCCTCCTCCTCTCACGG
CACGGCAGCTACGGGGGATTCCTTTCCCACCGCTCCTTCGCTTTC
CCTTCCTCGCCCGCCGTAATAAATAGACACCCCCTCCACACCCTC
TTTCCCCAACCTCGTGTTGTTCGGAGCGCACACACACACAACCAG
ATCTCCCCCAAATCCACCCGTCGGCACCTCCGCTTCAAGGTACGC
CGCTCGTCCTCCCCCCCCCCCCCTCTCTACCTTCTCTAGATCGGC
GTTCCGGTCCATGGTTAGGGCCCGGTAGTTCTACTTCTGTTCATG
TTTGTGTTAGATCCGTGTTTGTGTTAGATCCGTGCTGCTAGCGTT
CGTACACGGATGCGACCTGTACGTCAGACACGTTCTGATTGCTAA
CTTGCCAGTGTTTCTCTTTGGGGAATCCTGGGATGGCTCTAGCCG
TTCCGCAGACGGGATCGATTTCATGATTTTTTTTGTTTCGTTGCA
TAGGGTTTGGTTTGCCCTTTTCCTTTATTTCAATATATGCCGTGC
ACTTGTTTGTCGGGTCATCTTTTCATGCTTTTTTTTGTCTTGGTT
GTGATGATGTGGTCTGGTTGGGCGGTCGTTCTAGATCGGAGTAGA
ATTCTGTTTCAAACTACCTGGTGGATTTATTAATTTTGGATCTGT
ATGTGTGTGCCATACATATTCATAGTTACGAATTGAAGATGATGG
ATGGAAATATCGATCTAGGATAGGTATACATGTTGATGCGGGTTT
TACTGATGCATATACAGAGATGCTTTTTGTTCGCTTGGTTGTGAT
GATGTGGTGTGGTTGGGCGGTCGTTCATTCGTTCTAGATCGGAGT
AGAATACTGTTTCAAACTACCTGGTGTATTTATTAATTTTGGAAC
TGTATGTGTGTGTCATACATCTTCATAGTTACGAGTTTAAGATGG
ATGGAAATATCGATCTAGGATAGGTATACATGTTGATGTGGGTTT
TACTGATGCATATACATGATGGCATATGCAGCATCTATTCATATG
CTCTAACCTTGAGTACCTATCTATTATAATAAACAAGTATGTTTT
ATAATTATTTTGATCTTGATATACTTGGATGATGGCATATGCAGC
AGCTATATGTGGATTTTTTTAGCCCTGCCTTCATACGCTATTTAT
TTGCTTGGTACTGTTTCTTTTGTCGATGCTCACCCTGTTGTTTGG
TGTTACTTCTGCAA
SEQ ID NO: 4 is IQX₁YLDENKX₂LI and:
X₁ is K or Q
X₂ is K or Q or S
SEQ ID NO: 5 is LQX₁NLX₂YLAAIADX₃Q and:
X₁ is K, Q, H or R
X₂ is M or L
X₃ is A, T or S
SEQ ID NO: 6
Amino acid sequence of the Arabidopsis thaliana
AN3 orthologue (GIF2)
MQQQQSPQMFPMVPSIPPANNITTEQIQKYLDENKKLIMAIMENQ
NLGKLAECAQYQALLQKNLMYLAAIADAQPPPPTPGPSPSTAVAA
QMATPHSGMQPPSYFMQHPQASPAGIFAPRGPLQFGSPLQFQDPQ
QQQQIHQQAMQGHMGIRPMGMTNNGMQHAMQQPETGLGGNVGLRG
GKQDGADGQGKDDGK
SEQ ID NO: 7
Amino acid sequence of the Arabidopsis thaliana
AN3 orthologue (GIF3)
MQQSPQMIPMVLPSFPPTNNITTEQIQKYLDENKKLIMAILENQN
LGKLAECAQYQALLQKNLMYLAAIADAQPQPPAATLTSGAMTPQA
MAPNPSSMQPPPSYFMQQHQAVGMAQQIPPGIFPPRGPLQFGSPH
QFLDPQQQLHQQAMQGHMGIRPMGLNNNNGLQHQMHHHETALAAN
NAGPNDASGGGKPDGTNMSQSGADGQGGSAARHGGGDAKTEGK
SEQ ID NO: 8
Amino acid sequence of the Arabidopsis thaliana
AN3 gene (GIF1)
MQQHLMQMQPMMAGYYPSNVTSDHIQQYLDENKSLILKIVESQNS
GKLSECAENQARLQRNLMYLAAIADSQPQPPSVHSQYGSAGGGMI
QGEGGSHYLQQQQATQQQQMTQQSLMAARSSMLYAQQQQQQQPYA
TLQHQQLHHSQLGMSSSSGGGGSSGLHILQGEAGGFHDFGRGKPE
MGSGGGGEGRGGSSGDGGETLYLKSSDDGN
SEQ ID NO: 9
Amino acid sequence of the Oryza sativa
orthologous AN3 gene
(OsR498G0306998000.01)
MQQQHLMQMNQGMMGGYASPTTVTTDLIQQYLDENKQLILAILDN
QNNGKVEECARNQAKLQHNLMYLAAIADSQPPQTAAMSQYPSNLM
MQSGARYMPQQSAQMMAPQSLMAARSSMMYAQPALSPLQQQQQQA
AAAHGQLGMGSGGTTSGFSILHGEASMGGGGGGGGAGNSMMNAGV
FSDFGRGGGGGGSGGKEGSTSLSVDVRGANSGAQSGDGEYLKGTE
EEGS
SEQ ID NO: 10
Amino acid sequence of the Oryza sativa
orthologous AN3 gene
(OsR498G1120506400.01)
MQQQMAMPAGAAAAAVPPAAGITTEQIQKYLDENKQLILAILENQ
NLGKLAECAQYQAQLQKNLLYLAAIADAQPPQNPGSRPQMMQPGA
TPGAGHYMSQVPMFPPRTPLTPQQMQEQQQQQLQQQQAQALAFPG
QMLMRPGTVNGMQSIPVADPARAADLQTAAPGSVDGRGNKQDATS
EPSGTESHKSAGADNDAGGDIAEKS
SEQ ID NO: 11
Amino acid sequence of the Oryza sativa
orthologous AN3 gene
(OsR498G1221661000.01)
MQQQPMPMPAQAPPTAGITTEQIQKYLDENKQLILAILENQNLGK
LAECAQYQAQLQKNLLYLAAIADTQPQTTISRPQMVPHGASPGLG
GQYMSQVPMFPPRTPLTPQQMQEQQLQQQQAQLLSFGGQMVMRPG
VVNGIPQLLQGEMHRGADHQNAGAATSEPSESHRSTGTENDGGSD
FGDQS
SEQ ID NO: 12
Amino acid sequence of the Triticum aestivum
orthologous AN3 gene
(TraesCS4A02G151200)
MQQAMSLPPGAVGAVSSPAGITTEQIQKYLDENKQLILAILENQN
LGKLAECAQYQAQLQKNLLYLAAIADAQPPQNPTSHPQMVQPGSM
QGAGHYMSQVPMFPPRTPLTPQQMQEQQHQQLQQQQAQALSFPAQ
VVMRPGTVNGMQQPMQAAGDLQPAAAPGGSKQDAAVAGASSEPSG
TKSHKNAGAEEVGADVAEQS
SEQ ID NO: 13
Amino acid sequence of the Triticum aestivum
orthologous AN3 gene
(TraesCS4A02G250600)
MQQQHLMQMNQSMMGGYASSTTVTTDLIQQYLDENKQLILAILDN
QNNGKVEECARNQAKLQQNLMYLAAIADSQPPQTASLSQYPSNIM
MQSGPRYMPQQSAQMMSPQSLMAARSSMMYAQQAMSPLQQQQQQQ
QQHQAASHGQLGMSSGATTGFNLLHGEASMGGGGGATGNSMMNAG
VFSDYGRGGSGAKEGSTSLSADARGANSGAHSGDGEYLKGTEEEG
S
SEQ ID NO: 14
Amino acid sequence of the Triticum aestivum
orthologous AN3 gene
(TraesCS4B02G064000)
MQQQHLMQMNQSMMGGYASSTTVTTDLIQQYLDENKQLILAILDN
QNNGKVEECARNQAKLQQNLMYLAAIADSQPPQTASLSQYPSNLM
MQSGPRYMQQQSAQMMSPQSLMAARSSMMYAQQAMSPLQQQQQQQ
HHQAAAHGQLGMSSGATTGENLLHGEASMGGGGGATGNSMMNASV
ESDYGRGGSGAKEGSTSLSADARGANSGAHSGDGEYLKGTEEEGS
SEQ ID NO: 15
Amino acid sequence of the Triticum aestivum
orthologous AN3 gene
(TraesCS4B02G158300)
MQQAMSLPPGAVGAVSPPAGITTEQIQKYLDENKQLILAILENQN
LGKLAECAQYQAQLQKNLLYLAAIADAQPPQNPASRPQMVQQPGS
MQGAGHYMSQVPMFPPRTPLTPQQMQEQQQAQALSFPAQMVMRPG
TINGMQLPMQAAGDLQPAAAPGGSKQDAVVAGASSEPSGTESHKK
AGAEKEVSADVAEQS
SEQ ID NO: 16
Amino acid sequence of the Triticum aestivum
orthologous AN3 gene
(TraesCS4D02G062900)
MQQQHLMQMNQSMMGGYASSTTVTTDLIQQYLDENKQLILAILDN
QNNGKVEECARNQAKLQQNLMYLAAIADSQPPQTASLSQYPSNLM
MQSGPRYMPQQSAQMMSPQSLMAARSSMMYAQQAMSPLQQQQQQQ
HQAAAHGQLGMSSGATTGFNLLHGEASMGGGGGGASGNSMMNAGV
FSDYGRGGSGAKEGSTSLSADARGANSGAHSGDGEYIKGTEEEGS
SEQ ID NO: 17
Amino acid sequence of the Triticum aestivum
orthologous AN3 gene
(TraesCS5A02G107700)
MQQAMPMPPAAAAPGMPPSAGLSTEQIQKYLDENKQLILAILENQ
NLGKLAECAQYQAQLQKNLLYLAAIADTQPQTTVSRPQMAPPSAS
PGAGHYMSQVPMFPPRTPLTPQQMQEQQLQQQQAQMLPFAGQMVA
RPGAVNGMPQAPQVEPAYAAGGASSEPSGTESHRSTGADNDGGSG
LADQS
SEQ ID NO: 18
Amino acid sequence of the Triticum aestivum
orthologous AN3 gene
(TraesCS5B02G114600)
MQQAMPMPPAAAAPGMPPSAGLSTEQIQKYLDENKQLILAILENQ
NLGKLAECAQYQAQLQKNLLYLAAIADTQPQTSVSRPQMAPPSAS
PGVGHYMSQVPMFPPRTPLTPQQMQEQQLQQQQAQMLPFAGQMVA
RPGAVNGMARAPQVEPAYAAGGASSEPSGTESHRSTGADNNGGSG
LADQS
SEQ ID NO: 19
Amino acid sequence of the Triticum aestivum
orthologous AN3 gene
(TraesCS5D02G116800)
MQQAMPMPAAAAAPGMPPSAGLSTEQIQKYLDENKQLILAILENQ
NLGKLAECAQYQAQLQKNLLYLAAIADTQPQTSVSRPQMAPPSAS
PGAGHYMSQVPMFPPRTPLTPQQMQEQQLQQQQAQMLPFAGQMVV
RPGAVNGMPHAPQVEPAYAAGGASSEPSGTESHRSTGADNDGGSG
LADQS
SEQ ID NO: 20
Amino acid sequence of the Zea mays orthologous
AN3 gene (ZmGIF4)
MQQPMHMQPQAPAITPAAGISTEQIQKYLDENKQLILAILENQNL
GKLAECAQYQSQLQKNLLYLAAIADAQPQTAVSRPQMAPPGGSPG
VGQYMSQVPMFPPRTPLTPQQMQEQQLQQQQAQLLNFSGQMVARP
GMVNGMAQSMQAQLPPGVNKQDAGGVASEPSGTESHRSTGGDDGG
SD
SEQ ID NO: 21
Amino acid sequence of the Zea mays orthologous
AN3 gene (ZmGIF1)
MQQQHLMQMNQNMMGGYTSPAAVTTDLIQQYLDENKQLILAILDN
QNNGKAEECERHQAKLQHNLMYLAAIADSQPPQTAPLSQYPSNLM
MQPGPRYMPPQSGQMMNPQSLMAARSSMMYAHPSLSPLQQQQAAH
GQLGMAPGGGGGGTTSGFSILHGEASMGGGGAGAGAGNNMMNAGM
FSGFGRSGSGAKEGSTSLSVDVRGGTSSGAQSGDGEYLKVGTEEE
GS
SEQ ID NO: 22
Amino acid sequence of the Zea mays orthologous
AN3 gene (ZmGIF3)
MPMQPQAPSMTPAAGITTEQIQKYLDENKQLILAILENQNLGKLA
ECAQYQSQLQKNLLYLAAIADAQPETAVSRPQMAPPGASPGVGQY
MSQVPMFPPRTPLTPQQMLEQQLQQQQAQLLNFSGQMVARPGMVN
GMLQAMQVQQAQPSPVMNRPDAGGIASEPSGTESHRTSTGGDNGG
GSD
SEQ ID NO: 23
Amino acid sequence of the Zea mays orthologous
AN3 gene (ZmGIF2)
MQQQMPMPPAPAAAAAAAPPAAGITTEQIQKYLDENKQLILAILE
NQNLGKLAECAQYQAQLQKNLLYLAAIADAQPQPPQNPAGRPQMM
QPGIVPGAGHYMSQVPMFPPRTPLTPQQMQEQQQQQQFQQQQQQV
QALTEPGQMVMRPGTINGMQQQQPMQADPARAAAELQQAAPIPAD
GRGSKQDTAGGASSEPSANESHKSATGADTEAGGDVAEKS

4. Non-Limiting Examples of AN3 Protein Sequences of Ortholoqous Genes which can be Used to Construct Chimeric Genes of the Invention

AN3 orthologous genes can be easily recognized in plants. For example, Arabidopsis thaliana has 3 orthologous GIF genes, Zea mays has 4 orthologous genes, Oryza sativa has 3 orthologous genes and Triticum aestivum has 8 orthologous genes. FIG. 8 depicts 2 conserved boxes (bold and underlined) between the orthologous AN3 protein sequences, the sequences of these boxes are depicted in SEQ ID NO: 4 and SEQ ID NO: 5.

SEQ ID NO: 6 to SEQ ID NO: 23 depict protein sequences of orthologous plant genes of SEQ ID NO: 8. SEQ ID NO: 2 depicts the cDNA sequence of the orthologous Zea mays AN3 gene which was used for cloning in example 1 and SEQ ID NO: 21 depicts its corresponding protein sequence.

5. Comparison of Non-Transformed Corn Plants with Corn Plants Comprising the Chimeric Gene in the Greenhouse

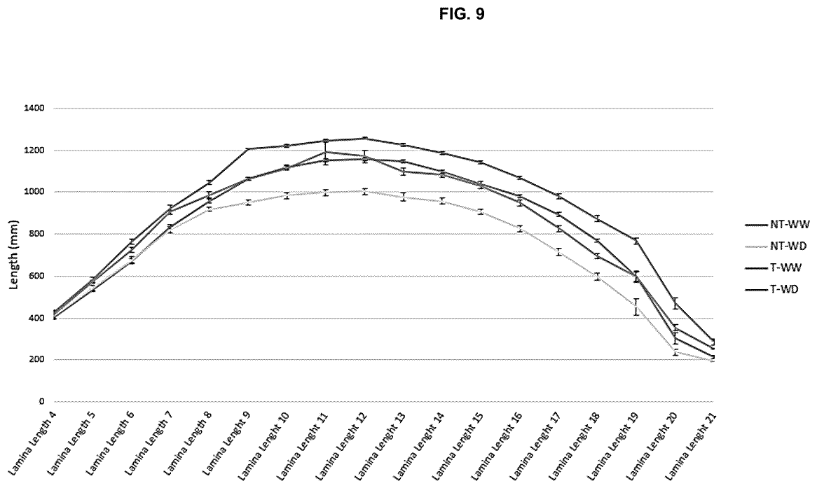

Non-transformed (NT) and transformed (T) corn plants were grown on an automated greenhouse-based phenotyping platform that controls the watering regime. In one condition corn plants were kept well-watered (WW, relative water content of 2.4) or in another condition plants experienced water deficit (WD) imposed at V5 by withholding water until relative water content of 1.4, after which daily watering was performed to maintain this water content. It was observed that the leaves 6-19 of the transgenic (T) plants were larger than those of the segregating non-transgenic (NT) plants, both in WW and WD conditions (see FIG. 9).

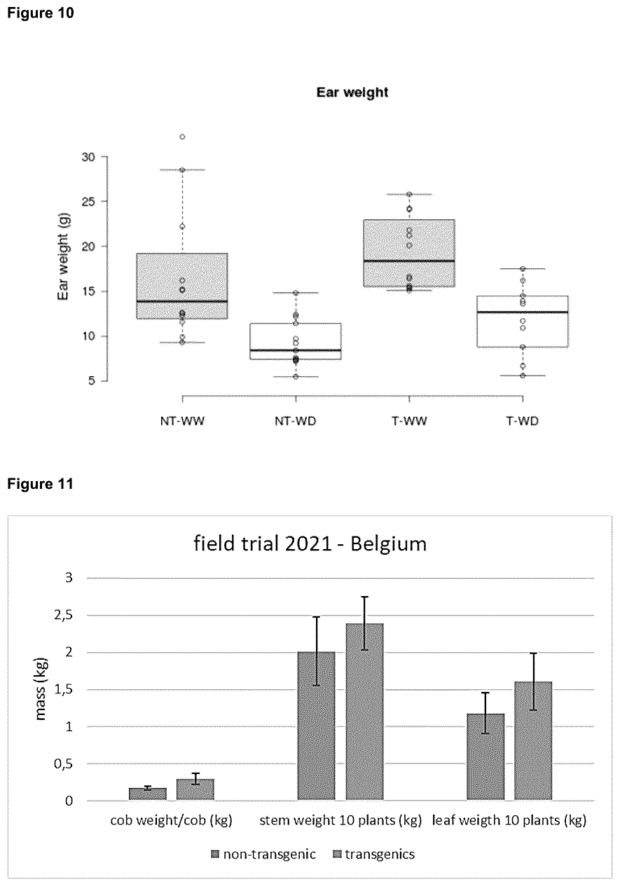

Non-transformed and transformed corn plants were grown on an automated greenhouse-based phenotyping platform that controls the watering regime. In one condition corn plants were kept well-watered (WW, relative water content of 2.4) or in another condition plants experienced water deficit (WD) imposed at V5 by withholding water until relative water content of 1.4, after which daily watering was performed to maintain this water content. The plants were harvested at silking, so before pollination. It was observed that the weight of the non-pollinated ears was higher in the transgenic (T) plants compared to the segregating non-transgenic (NT) plants, both in WW and WD conditions (see FIG. 10).

### Materials and Methods

### Phenotypic Measurements

Final plant height was measured by placing a ruler on the soil at the base of the stem and measuring the height of the highest leaf collar. Leaf lamina length was measured from the leaf collar to the leaf tip using a ruler. Leaf width was measured at the middle of the leaf (at the leaf length-axis) using a ruler. Cobs weight was analysed by removing the cobs from the plants followed by weighing the cobs using a scale. Spikelets on the cobs were analysed by first dehusking the ear, followed by counting the number of spikelet rows and counting spikelets per row.

1. A chimeric gene construct comprising the following operably linked DNA elements:
a) the promoter region of the Brachypodium distachyon elongation factor 1-alpha (EF1A) gene,
b) a DNA region encoding a plant AN3 protein comprising SEQ ID NO: 4 and SEQ ID NO: 5, and
c) a 3′ end region comprising transcription termination and polyadenylation signals functioning in cells of a plant.

2. The chimeric gene of claim 1, wherein the EF1A promoter comprises SEQ ID NO: 1.

3. The chimeric gene of claim 1, wherein the plant AN3 gene encodes a protein comprising SEQ ID NO: 6, 7, 8, 9, 10, 11, 12, 13, 14, 15, 16, 17, 18, 19, 20, 21, 22 or 23.

4. The chimeric gene construct of claim 1, wherein the chimeric gene construct is comprised in a vector.

5. The chimeric gene construct of claim 1, wherein the chimeric gene construct is comprised in a plant, plant cell or plant seed.

6. A method for increasing the yield of plants, the method comprising:
introducing or transforming a plant with the chimeric gene construct of claim 1, and
selecting a plant with a stable expression of the chimeric gene.

7. A method for increasing the drought tolerance of plants, the method comprising:
introducing or transforming a plant with the chimeric gene construct of claim 1, and
selecting a plant with a stable expression of the chimeric gene.

8. The method according to claim 6, wherein the plants are cereals.

9. The method according to claim 7, wherein the plants are cereals.
